(19) United States
(12) Patent Application Publication
(10) Pub. No.: US 2024/0423788 A1
(43) Pub. Date: Dec. 26, 2024

(54) Self-Closing Outer Cuff Using Valve Foreshortening

(71) Applicant: St. Jude Medical, Cardiology Division, Inc., St. Paul, MN (US)

(72) Inventor: Alexander Barrette, Maplewood, MN (US)

(73) Assignee: St. Jude Medical, Cardiology Division, Inc., St. Paul, MN (US)

(21) Appl. No.: 18/626,714

(22) Filed: Apr. 4, 2024

Related U.S. Application Data
(60) Provisional application No. 63/509,799, filed on Jun. 23, 2023.

(51) Int. Cl. A61F 2/24; A61F 2/95

(52) U.S. Cl. CPC A61F 2/2418; A61F 2/2436; A61F 2/9525; A61F 2220/0008

(57) ABSTRACT

A prosthetic heart valve includes a collapsible and expandable stent having a plurality of cells including a lowermost row of cells, a valve including an inner cuff, and a plurality of leaflets secured to the stent, and an outer cuff at least partially covering the lowermost row of cells, the outer cuff having a bottom edge, a top edge, and a plurality of axially projecting arms extending from the top edge, the outer cuff being attached to at least one cell at two side vertices and a bottom vertex to form at least one parachute, and to an upper vertex of the at least one cell via at least one of the plurality of axially projecting arms.

### CROSS-REFERENCE TO RELATED APPLICATIONS

The present application claims priority to U.S. Provisional Ser. No. 63/509,799, filed Jun. 23, 2023 the disclosure of which is hereby incorporated by reference in its entirety as if fully set forth herein.

### BACKGROUND OF THE DISCLOSURE

The present disclosure relates to prosthetic heart valve implantation and, more particularly, to improvements for loading a self-expanding collapsible heart valve into a delivery device.

Prosthetic heart valves may be formed from biological materials such as harvested bovine valves or pericardium tissue. Such valves are typically fitted within a stent, which may be inserted into the heart at the annulus of the compromised native valve to replace the native valve. Prosthetic heart valves that are collapsible to a relatively small circumferential size can be delivered into a patient less invasively than valves that are not collapsible. For example, a collapsible valve may be delivered into a patient via a tube-like delivery apparatus such as a catheter, a trocar, a laparoscopic instrument, or the like. To perform such insertion procedure, it is often necessary to compress the stent to a reduced diameter for loading into the delivery device.

In the case of prosthetic valves formed from biological materials, the stented valve is preferably preserved in the open condition for storage. The valve may be crimped or its diameter be reduced for loading in the delivery device, in the operating arena.

Present devices and methods for collapsing a stented valve may result in the outer cuff of the valve catching or snagging on an edge of the delivery device. The outer cuff snagging may produce peak loading and resheathing forces. It would therefore be beneficial to improve stented heart valves for easier crimping and loading. Such devices and methods would allow for a successful and efficient loading of the heart valve in the delivery device.

### BRIEF SUMMARY OF THE DISCLOSURE

According to an embodiment of the disclosure, a prosthetic heart valve includes a collapsible and expandable stent having a plurality of cells including a lowermost row of cells, a valve including an inner cuff, and a plurality of leaflets secured to the stent, and an outer cuff at least partially covering the lowermost row of cells, the outer cuff having a bottom edge, a top edge, and a plurality of axially projecting arms extending from the top edge, the outer cuff being attached to at least one cell at two side vertices and a bottom vertex to form at least one parachute, and to an upper vertex of the at least one cell via at least one of the plurality of axially projecting arms.

According to an embodiment of the disclosure, a method of loading a prosthetic heart valve includes providing a prosthetic heart valve having a collapsible and expandable stent having a plurality of cells including a lowermost row of cells, a valve including an inner cuff, and a plurality of leaflets secured to the stent, and an outer cuff at least partially covering the lowermost row of cells, the outer cuff having a bottom edge, a top edge, and a plurality of axially projecting arms extending from the top edge, the outer cuff being attached to at least one cell at two side vertices and a bottom vertex to form at least one parachute, and to an upper vertex of the at least one cell via at least one of the plurality of axially projecting arms, axially stretching the collapsible and expandable stent so that the plurality of projecting arms gathers the outer cuff closer to the stent, and loading the prosthetic heart valve into a delivery device.

### BRIEF DESCRIPTION OF THE DRAWINGS

Various embodiments of the present loading assembly are disclosed herein with reference to the drawings, wherein:

FIG. 1 is a perspective view of a distal portion of a prior art delivery device;

FIG. 2 is a perspective view of a proximal portion of the delivery device of FIG. 1;

FIG. 3 is a perspective view of a prior art embodiment of a collapsible prosthetic heart valve in an expanded condition;

FIG. 4 is a front view of another embodiment of a collapsible prosthetic heart valve in an expanded condition;

FIG. 5 is a perspective view of a loading funnel;

FIG. 6 is a schematic longitudinal cross-section of the loading funnel of FIG. 5, illustrating a coating applied to the inner surface thereof;

FIG. 7 is a perspective view of a loading base for use with the loading funnel of FIG. 5;

FIG. 8 illustrates the expanded prosthetic heart valve of FIG. 3 assembled onto the loading base of FIG. 7;

FIG. 9 illustrates the loading funnel of FIG. 5 being coupled to the loading base of FIG. 8 for collapsing the prosthetic heart valve of FIG. 8;

FIGS. 10A-B are schematic illustrations showing a prosthetic heart valve with a flared outer cuff and a gathered outer cuff, respectively;

FIG. 11 is a schematic representation of one example of an outer cuff according to an embodiment of the present disclosure;

FIG. 12 illustrates a cell of a stent in relaxed and stretched conditions; and

FIG. 13 is a schematic illustration showing the cuff of FIG. 11 being coupled to the outer diameter of a stent.

### DETAILED DESCRIPTION

Embodiments of the presently disclosed loading assemblies and heart valves are described herein in detail with reference to the drawing figures, wherein like reference numerals identify similar or identical elements. In the drawings and in the description which follows, the term “proximal” refers to the end of the loading assembly, or portion thereof, which is closest to the operator during use, while the term “distal” refers to the end of the loading assembly, or portion thereof, which is farthest from the operator during use.

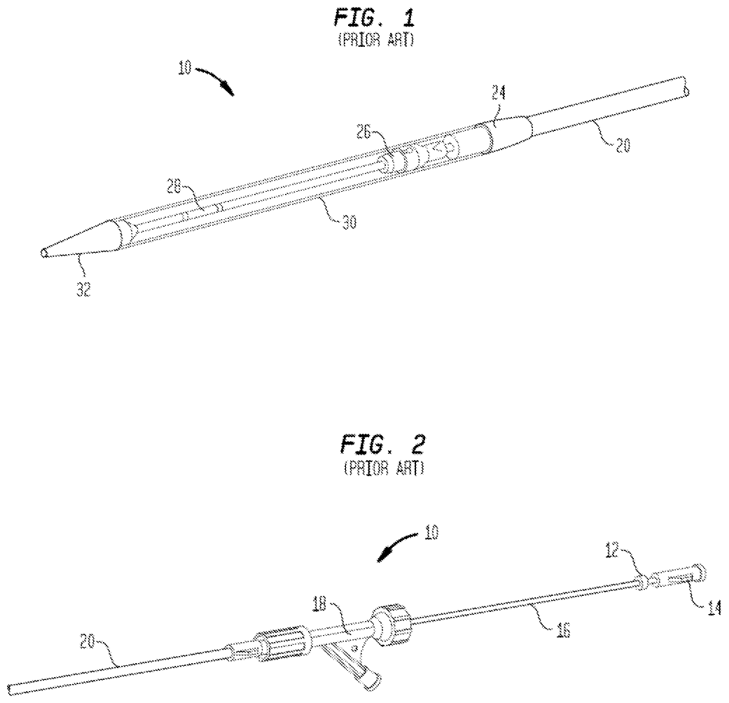

The present disclosure relates to assemblies and methods for loading a self-expanding stent or a collapsible prosthetic heart valve into a minimally invasive delivery device. An exemplary minimally invasive delivery device 10 is illustrated in FIGS. 1 and 2.

As seen in FIG. 2, the delivery device 10 may include an inner tube 16 having a lumen extending therethrough. A hub 14 is mounted on the proximal end of the inner tube 16 and is adapted for connection to another system or mechanism, such as a handle, a syringe or a mechanism for displacing a distal sheath 30. Mechanisms for displacing the distal sheath 30 are described in International Patent Application Publication No. WO/2009/091509, the entire contents of which are hereby incorporated herein by reference. A retention ring 12 may also be mounted on the proximal end of the inner tube 16.

As shown in FIG. 1, an outer shaft 20 of the delivery device 10 extends to a transition member 24, which may have a tapered shape. The transition member 24 interconnects a distal end of the outer shaft 20 and the distal sheath 30. The distal sheath 30 surrounds a retaining element 26 and a support shaft 28 and can maintain a prosthetic heart valve mounted around the support shaft in a collapsed condition. The support shaft 28 is operatively connected to the inner tube 16 and has a lumen extending therethrough for receiving a guidewire (not shown). The retaining element 26 is mounted on the support shaft 28 and is configured for supporting an end of a prosthetic heart valve or any other suitable medical implant. The retaining element 26 may be longitudinally and rotatably fixed relative to the support shaft 28, thereby preventing the cells of the stent from entangling with one another during deployment. The distal sheath 30 covers the retaining element 26 and at least a portion of the support shaft 28 and is movable relative to the support shaft between a distal position shown in FIG. 1 and a proximal position (not shown). An atraumatic tip 32 may be connected to the distal end of the support shaft 28, and may have a tapered shape.

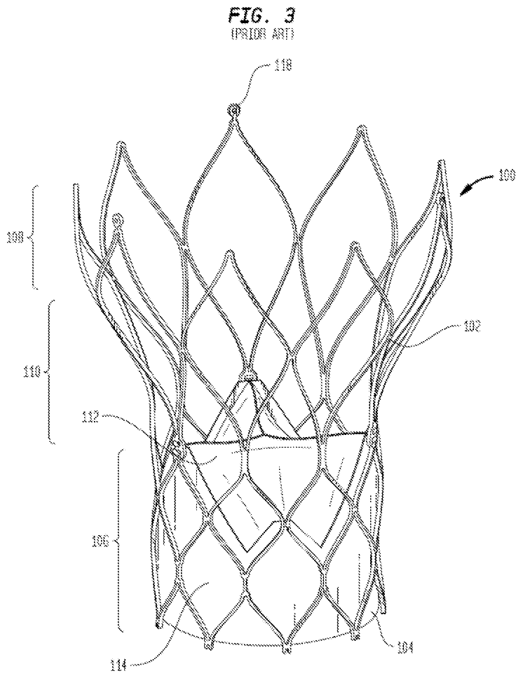

FIG. 3 shows one embodiment of a prosthetic valve 100 designed to replace a native aortic valve. The valve 100 has a collapsed condition and an expanded condition and may be formed from a collapsible framework or stent 102, with a valve assembly 104 internally connected to the stent. The stent 102 may be formed from any suitable biocompatible material, such as nitinol, and may include an annulus section 106, an aortic section 108, and an intermediate section 110. The aortic section 108 may have a larger diameter than the annulus section 106 in the expanded condition. The intermediate section 110 of the stent 102 is located between the annulus section 106 and the aortic section 108. A plurality of blunted horseshoe ends may be disposed at the proximal edge of annulus section 106. The valve assembly 104 may include a plurality of leaflets 112 and an inner cuff 114 attached to the stent 102. The leaflets 112 and the inner cuff 114 may be formed from a biocompatible polymer, from bovine or porcine pericardial tissue, or from other appropriate biocompatible materials. The valve assembly 104 is connected to the stent 102 generally within the annulus section 106, but may extend into the intermediate section 110. The valve 100 may include tabs or retaining members 118 at spaced positions around one or both ends of the stent 102. The retaining members 118 are typically designed to mate with pockets (not shown) in retaining element 26 to maintain the prosthetic valve 100 in assembled relationship with the delivery device 10, to minimize longitudinal movement of the prosthetic valve relative to the delivery device during unsheathing and resheathing procedures, to help prevent rotation of the prosthetic valve relative to the delivery device as the delivery device is advanced to the target site and during deployment, and to maintain the alignment of the stent cells and prevent them from becoming tangled.

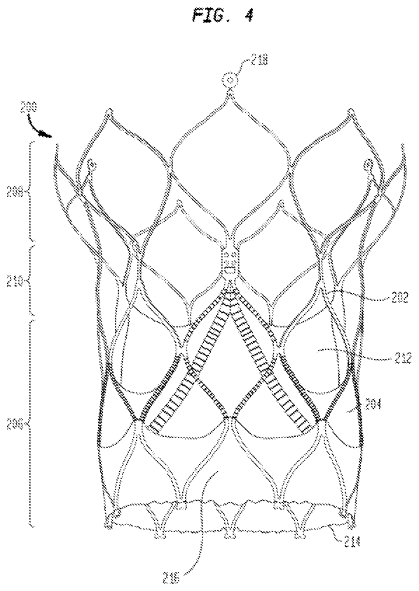

FIG. 4 shows another embodiment of a prosthetic valve 200 designed to replace a native aortic valve. The valve 200 may be similar in construction to the valve 100 described above and may be formed from a collapsible framework or stent 202, with a valve assembly 204 internally connected to the stent. The stent 202 may include an annulus section 206, an aortic section 208, and an intermediate section 210. The aortic section 208 may have a larger diameter than the annulus section 206 in the expanded condition. The intermediate section 210 of the stent 202 is located between the annulus section 206 and the aortic section 208. The valve assembly 204 may include a plurality of leaflets 212 and an inner cuff 214 attached to the stent 202. The valve 200 further includes an outer cuff 216 attached to the annulus section 206. More examples of outer cuffs are described in U.S. Pat. No. 8,808,356, the entire content of which is hereby incorporated herein by reference. The outer cuff 216 promotes sealing with native tissue even where the native tissue is irregular.

The prosthetic valves 100, 200 are preferably stored in their expanded or open condition. As such, the valves 100, 200 may be crimped into a collapsed or reduced diameter condition for surgical implantation. The crimping process is preferably conducted in the operating arena by the surgeon, interventional cardiologist or surgical assistant using a specialized assembly.

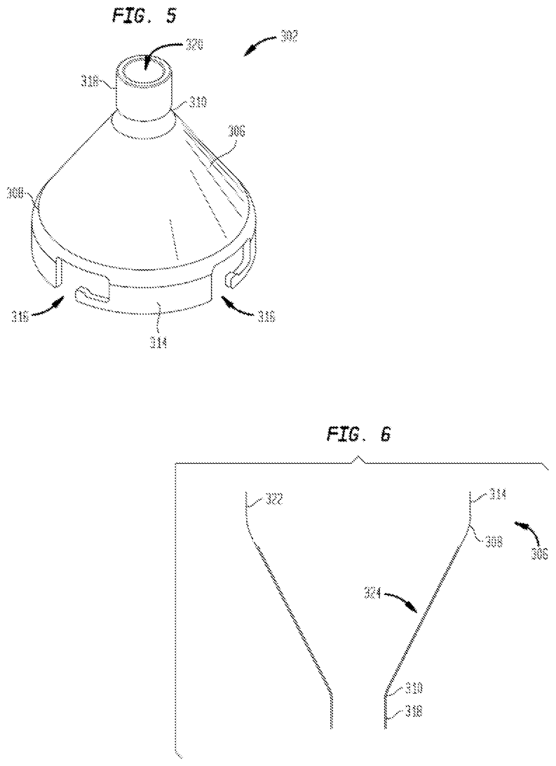

Some exemplary loading assemblies for loading the prosthetic valve 200 into a delivery device are described in U.S. Pat. Nos. 9,021,674; 8,931,159; and 8,893,370, the entire contents of which are hereby incorporated herein by reference. Referring now to FIGS. 5-7, a loading assembly according to an embodiment of the present disclosure is illustrated. The loading assembly generally includes a compression member 302 and a loading base 404, both adapted to be coupled to one another. The compression member 302 includes a funnel 306 having a substantially frusto-conical shape with a larger diameter at a first end 308 and a smaller diameter at a second end 310. The diameter of the funnel 306 may decrease either uniformly or non-uniformly from the first end 308 to the second end 310 to compress the valve 200 as the valve is advanced through the compression member 302. The compression member 302 is preferably made of a substantially rigid material, and may be wholly or partly made of a transparent plastic, such as polycarbonate or acrylic, to allow viewing of the valve 200 during loading.

The compression member 302 may further include an annular rim 314 extending from the first end 308 of the funnel 306 for joining the compression member to the loading base 404 as described below. The rim 314 may include a plurality of slots 316 disposed around its outer periphery. While the drawings show slots 316 that are substantially P-shaped, the slots may have any other shapes suitable for securely holding the compression member 302 to the loading base 404. The rim 314 may include four such slots 316, or more or less than four. Regardless of the number or slots 316, adjacent slots are preferably spaced equidistantly from each other.

The compression member 302 also may include a tubular extension 318 projecting from the second end 310 of the funnel 306. The tubular extension 318 has an opening 320 therethrough in communication with the interior of funnel 306. The opening 320 is sized and shaped to receive the distal sheath 30 of the delivery device 10 therein. The cross-section of the tubular extension 318 is preferably substantially circular, but may be oblong, oval, elliptical, or polygonal.

FIG. 6 depicts a schematic longitudinal cross-section of the compression member 302, showing the inner surface 322 thereof. In an exemplary embodiment, at least a portion of the inner surface 322 is coated with a layer 324 of a hydrophilic coating (HPC). By way of non-limiting examples, the HPC may include lubricious coatings available under the trade mark Serene™ from Surmodics, Inc. of Eden Prairie, MN. The layer 324 serves to reduce friction between the inner surface 322 and the valve 200, including the stent 202 and the outer cuff 216.

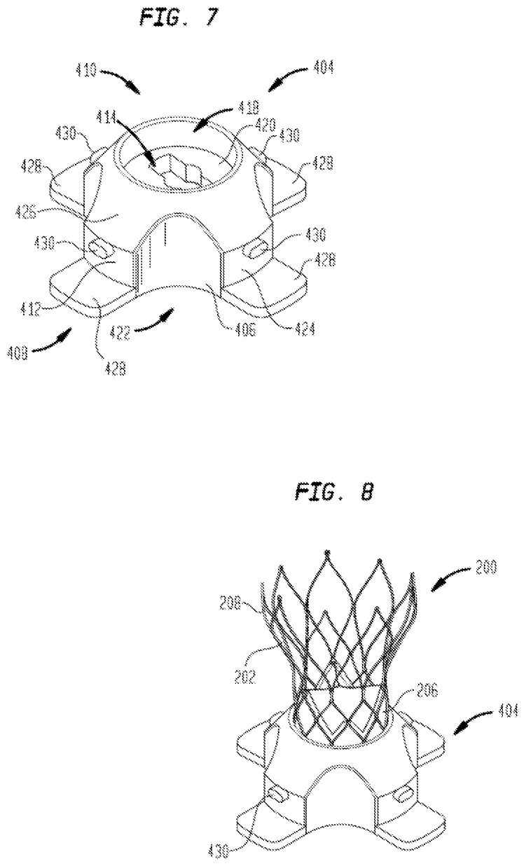

Referring to FIG. 7, the loading base 404 is preferably made in whole or in part of a substantially rigid material, and includes a body 406 having a substantially flat or planar bottom support surface 408 and a top end 410. The body 406 has an outer wall 412 and an aperture 414 extending axially through substantially the center of the body. The aperture 414 is sized to receive at least a portion of the tip 32 of the delivery device 10 therein. A recess 418 extends downwardly from the top end 410 of the body 406 concentrically with the aperture 414 so as to define a support surface 420 at a spaced distance from the top end. The recess 418 has a diameter and a depth defined by the support surface 420 sufficient to receive at least a portion of the annulus section 206 of the stent 202 in a fully or almost fully expanded condition.

The outer wall 412 of the body 406 does not extend continuously around the body, but rather may be interrupted by a plurality of inwardly curved indentations 422 which divide the outer wall into a plurality of wall segments 424, only two of which are shown in FIG. 7. Although FIG. 7 depicts a loading base 404 having four indentations 422 evenly spaced around the periphery of the body 406, it is contemplated that the loading base may be provided with more or less than four such indentations. Indentations 422 facilitate the grasping of loading base 404.

The outer wall segments 424 of the body 406 do not extend all the way to the top end 410 of the body, but rather terminate at their top ends at a continuous wall 426 oriented at an oblique angle to the outer wall 412. At their bottom ends, outer wall segments 424 each include a radially projecting supporting plate 428, the bottom surfaces of which are substantially coplanar with the bottom support surface 408 of the body 406. At least one pin 430 may protrude radially outward from each outer wall segment 424. The pins 430 are preferably spaced a sufficient distance from supporting plates 428 and sized and shaped to be received in the slots 316 of the compression member 302 to join the compression member and the loading base 404 together. When joined together, the compression member 302 and the loading base 404 collectively define a partial loading assembly.

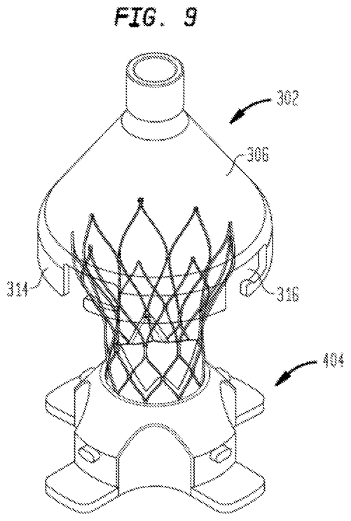

The loading assembly described above may be used to load the collapsible prosthetic heart valve 200 into a delivery device. As shown in FIG. 8, with the loading base 404 on a flat surface, at least a portion of the annulus section 206 of the stent 202 may be placed within the recess 418 of the loading base until the end of the stent contacts support surface 420. The compression member 302 may then be placed over the aortic section 208 of the stent 202 so that the aortic section of the stent is positioned within the funnel 306, as depicted in FIG. 9. The compression member 302 and the loading base 404 may then be pushed together, the tapered inner surface 322 of the funnel 306 gradually compressing the valve 200 until a portion of the aortic section 208 of the stent 202 is forced into and through the opening 320 of the compression member. When the portion of the aortic section 208 of the stent 202 passes through the opening 320 of the compression member 302, the retainers 218 of the stent will protrude through the opening 320 and will be positioned closely adjacent to one another. At this point, the pins 430 of the loading base 404 will be positioned within the slots 316 of the compression member 302, and the members may be locked together by rotating the loading base relative to the compression member, such that the pins 430 of the loading base slide toward the closed ends of the slots 316 of the compression member.

In some embodiments, it may be possible to improve a heart valve by reducing or eliminating potential snag of the outer cuff during valve loading and resheathing. As previous noted, outer cuff snagging may produce peak loading and resheathing forces. By reducing the snag, higher bulk cuffs that include larger parachutes may be loaded and resheathed into existing delivery systems, leading to improved paravalvular leakage performance in-vivo. Alternatively, existing outer cuff configurations may be loaded and resheathed with reduced force.

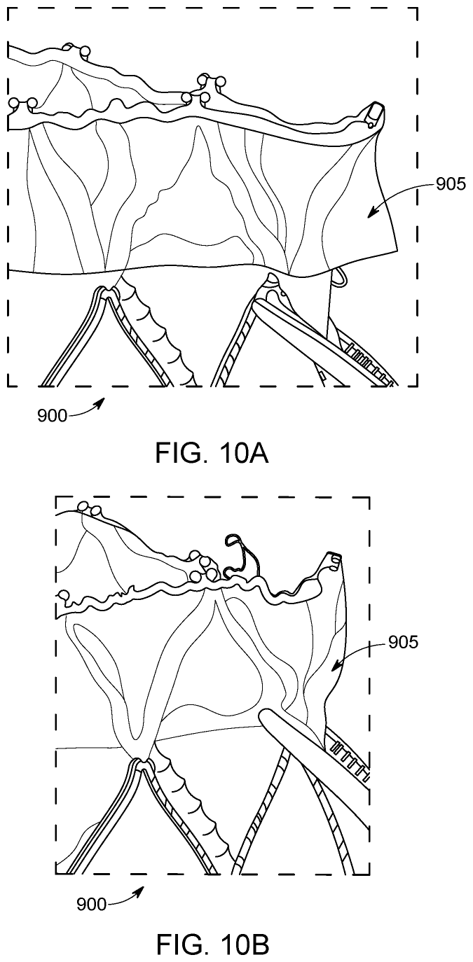

FIGS. 10A-B show two illustrations of a heart valve 900. Due to the active nature of the outer cuff for certain prosthetic heart valves, such as those used to prevent or reduce paravalvular leakage, the outer cuff 905 may flare or fold outside of the stent as shown in FIG. 10A. Though outward billowing of the outer cuff 905 is an expected and useful feature when implanted, it presents a challenge during loading. One challenge is that the folds of cuff 905 may snag on the distal outer member of the delivery system, increasing the total loading force. Alternatively, outer cuff 905 may be folded inwards (FIG. 10B), minimizing the material remaining on the outer circumference of the stent. As the outer cuff is pulled into the delivery system, the reduced material on the outer circumference of the stent may reduce the overall loading forces by reducing the snagging effect observed. Thus, one objective of the present disclosure is to reduce loading forces by biasing or urging the outer cuff to fold into the stent (FIG. 10B) instead of outside of the stent (FIG. 10A).

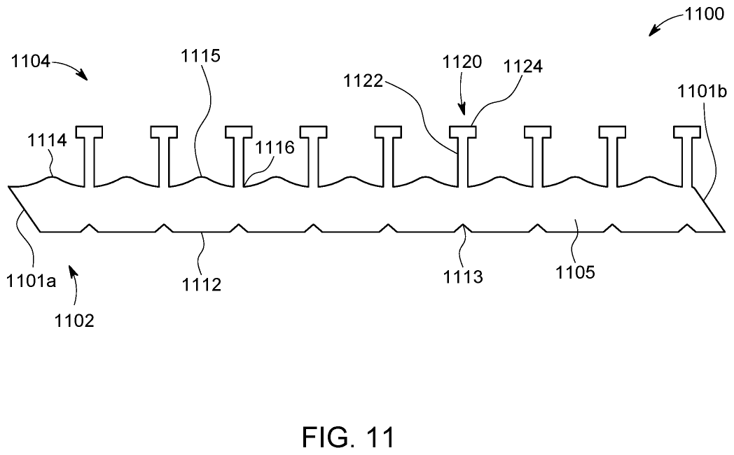

FIG. 11 illustrates an outer cuff 1100 configured to improve control of the movement of the outer cuff during loading and resheathing. Outer cuff 1100 includes a body 1105 that generally extends between a proximal end 1102 and a distal end 1104. In this example, body 1105 is unitary (i.e., the outer cuff is formed of a single continuous sheet that is wrapped so that side edges 1101a, 1101b are disposed adjacent one another, and possibly coupled together). It will be understood that body 1105 may be divided into two, three or more segments that are attached together. A bottom edge 1112 is disposed adjacent proximal end 1102 and a top edge 1114 is disposed adjacent distal end 1104. As shown, bottom edge 1112 include a series of spaced notches 1113 configured to couple to the blunted terminal ends of the stent. The top edge 1114 may include alternating peaks 1115 and valleys 1116. The top edge 1114 may also include a plurality of arms 1120 intended to be sewn to the top of the stent cell directly above the middle of a parachuting or billowing paravalvular leakage component. In the example shown, each arm 1120 include an elongated axial extension 1122 and a transverse terminal end 1124 so that the arm 1120 is T-shaped. Alternative shapes and/or sizes are also possible. Arms 1120 may connect to stent 102 and control the motion of the center of the parachute via the increasing cell height as the stent collapses into a delivery capsule.

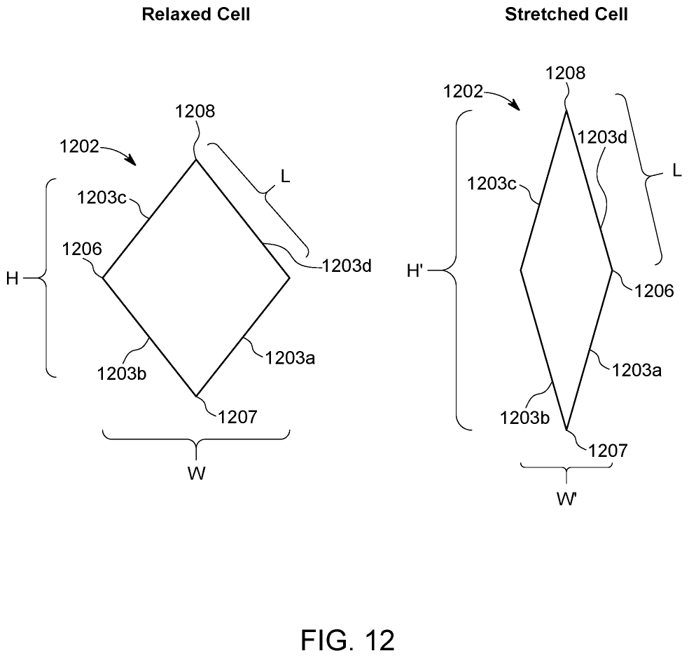

Outer cuff 1100 may be coupled to a stent 102 having a plurality of cells 1202. In some examples, stent 102 has a plurality of substantially diamond-shaped cells that can transition from a relaxed condition (e.g., at rest when no radial forces are applied thereto) to a stretched condition (e.g., for delivery or loading when external radial forces are applied thereto) and vice versa. FIG. 12 illustrates one such cell 1202 that includes four struts 1203a-d. In this examples, two struts 1203c and 1203b are joined at side vertex 1206, and two struts 1203a, 1203d are joined at an opposing side vertex. Struts 1203b and 1203a may also be joined at lower vertex, and struts 1203c and 1203d are joined together at an upper vertex. As shown, the shape of the cell will change when moving from the relaxed condition to the stretched condition. Specifically, when stretched, the cell will elongate to a height H′ that is greater than the original height H. Conversely, the cell will narrow when stretching to a width W′ that is less than the original width W.

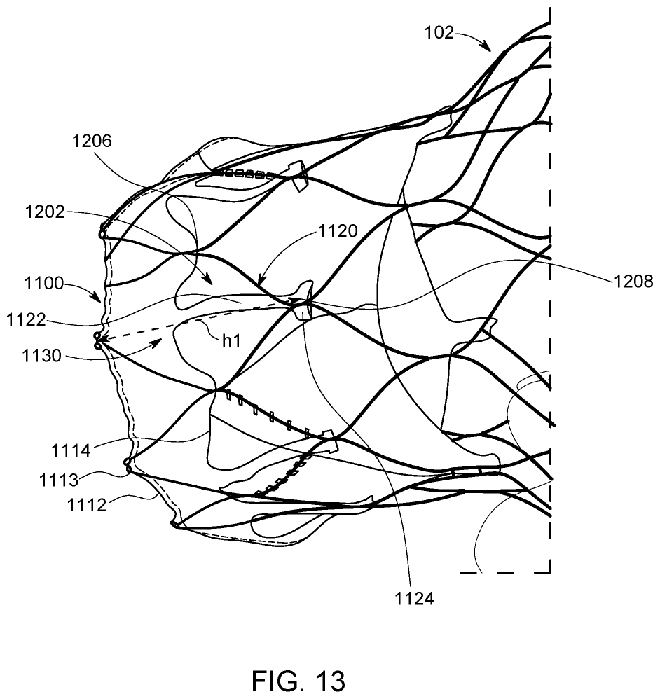

FIG. 13 is a schematic illustration provided to show one example of attaching outer cuff 1100 to stent 102. As shown, outer cuff 1100 is wrapped around the outer diameter of stent 102 with bottom edge 1112 of the outer cuff being disposed at a proximal-most end of the stent, and coupled thereto. The lowermost row of cells of stent 102 may include substantially diamond-shaped cells 1202 similar to those described in FIG. 12. In this example, outer cuff 1110 may be coupled to two opposing side vertices 1206 of a cell with sufficient slack so that parachutes or billowing portions 1130 are formed over a cell 1202 of stent 102. Outer cuff 1110 may also be coupled at an upper vertex 1208 of cell 1202 via arms 1120, the upper vertex 1208 being disposed opposite the blunt end of the stent. In some examples, transverse terminal end 1124 of the outer cuff 1100 wraps around struts of the stent that form upper vertex 1206 and is secured thereto via one or more sutures. In some examples, axial extension 1122 spans approximately (¼, ⅓, or ½ of a cell). In some examples, arms 1120 form approximately ¼, ⅓, ½, or ⅔ of the overall the height of the outer cuff. In some examples, the height h1 of the outer cuff 1110 from notch 1113 to the top of arm 1120 is substantially equal to the length of a lowermost cell 1202, or slightly less than the length of the lowermost cell 1202.

In use, as the stent transitions to a stretched condition (e.g., during crimping and/or loading), the stent cells collapse width-wise while increasing the vertical length of the cell. By setting the height h1 of the outer cuff 1110 equal to, or slightly less than the length of the elongated cell, arm 1120 may pull taught and fold outer cuff 1110 inwardly. As the valve is deployed, the cell foreshortens and arms 1120 become slack, allowing the outer cuff to billow outwardly to function as intended. It will be understood that arms 1120 may include a single arm sewn at the center of the parachute (or aligned with the center of a cell), or multiple arms for a single parachute 1130 connecting to a single point. Arms 1120 may be formed as a continuous or unitary extension of an existing outer cuff material and laser cut into the desired pattern. Alternatively, arms 1120 may be separately formed of a fabric swatch that is attached to the stent and outer cuff.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A prosthetic heart valve comprising:
a collapsible and expandable stent having a plurality of cells including a lowermost row of cells;
a valve including an inner cuff, and a plurality of leaflets secured to the stent; and
an outer cuff at least partially covering the lowermost row of cells, the outer cuff having a bottom edge, a top edge, and a plurality of axially projecting arms extending from the top edge, the outer cuff being attached to at least one cell at two side vertices and a bottom vertex to form at least one parachute, and to an upper vertex of the at least one cell via at least one of the plurality of axially projecting arms.

2. The prosthetic heart valve according to claim 1, wherein the lowermost row of cells includes a first quantity of cells, and the plurality of axially projecting arms includes a first quantity of arms equal to the first quantity of cells.

3. The prosthetic heart valve according to claim 2, wherein the lowermost row of cells includes nine cells, and the plurality of axially projecting arms includes nine arms.

4. The prosthetic heart valve according to claim 1, wherein each of the plurality of axially projecting arms is T-shaped.

5. The prosthetic heart valve according to claim 1, wherein each of the plurality of axially projecting arms includes an axial extension and a transverse terminal end.

6. The prosthetic heart valve according to claim 5, wherein the axial extension of each of the plurality of axially projecting arms is approximately half a length of the at least one cell when the at least one cell is in a stretched condition.

7. The prosthetic heart valve according to claim 5, wherein each of the plurality of axially projecting arms is approximately equal in length to the at least one cell when the at least one cell is in a stretched condition.

8. The prosthetic heart valve according to claim 7, wherein each of the plurality of axially projecting arms is approximately greater in length to the at least one cell when the at least one cell is in a relaxed condition.

9. The prosthetic heart valve according to claim 5, wherein each of the plurality of axially projecting arms is approximately shorter in length to the at least one cell when the at least one cell is in a stretched condition.

10. The prosthetic heart valve according to claim 9, wherein each of the plurality of axially projecting arms is approximately greater in length to the at least one cell when the at least one cell is in a relaxed condition.

11. The prosthetic heart valve according to claim 5, wherein the transverse terminal end is coupled to the upper vertex of the at least one cell via a suture.

12. The prosthetic heart valve according to claim 5, wherein the transverse terminal end is wrapped around the upper vertex of the at least one cell.

13. The prosthetic heart valve according to claim 1, wherein the outer cuff includes a plurality of spaced notches along the bottom edge, and wherein each of the plurality of axially projecting arms is aligned with one of the plurality of spaced notches.

14. A method of loading a prosthetic heart valve comprising:
providing a prosthetic heart valve including a collapsible and expandable stent having a plurality of cells including a lowermost row of cells, a valve including an inner cuff, and a plurality of leaflets secured to the stent, and an outer cuff at least partially covering the lowermost row of cells, the outer cuff having a bottom edge, a top edge, and a plurality of axially projecting arms extending from the top edge, the outer cuff being attached to at least one cell at two side vertices and a bottom vertex to form at least one parachute, and to an upper vertex of the at least one cell via at least one of the plurality of axially projecting arms;
axially stretching the collapsible and expandable stent so that the plurality of projecting arms gathers the outer cuff closer to the stent; and
loading the prosthetic heart valve into a delivery device.
